(19) United States
(12) Patent Application Publication
(10) Pub. No.: US 2015/0257889 A1
(43) Pub. Date: Sep. 17, 2015

(54) FEMORAL COMPONENT FOR A FEMORAL KNEE IMPLANT SYSTEM

(71) Applicant: Hyung Wook KANG, Los Altos, CA (US)

(72) Inventor: Hyung Wook Kang, Los Altos, CA (US)

(21) Appl. No.: 14/441,374

(22) PCT Filed: Nov. 7, 2012
(86) PCT No.: PCT/US12/63858

§ 371 Date: May 7, 2015

(51) Int. Cl. A61F 2/38

(52) U.S. Cl. CPC A61F 2/3859; A61F 2/3868; A61F 2/3886

(57) ABSTRACT

A femoral knee replacement prosthesis is disclosed including a femoral component, a tibial bearing component, and a tibial platform component. The femoral component includes an anterior condyle with a proximal lateral aspect adjacent a proximal medial aspect and separated by a patella groove, a distal lateral aspect adjacent a distal condyle medial aspect, and a lateral posterior condyle parallel with a medial posterior condyle. The distal condyle lateral and medial aspects are inferior the proximal lateral and medial aspects and the lateral and medial posterior condyles extend posteriorly from the distal condyle lateral and medial aspects. The tibial bearing component includes a proximal side for mating with the femoral component and a distal side. The tibial platform component includes a proximal side with an opening for receiving the tibial bearing component and a distal side including a post adapted to be fixed in a tibia.

### FIELD OF THE INVENTION

The present invention relates to devices, systems, and methods for total knee arthroplasty. The present invention includes a femoral knee replacement prosthesis with mobile bearing technology.

### BACKGROUND OF THE INVENTION

Prior knee replacement prosthesis inventions are not adequate for all patients, because they are based on a Western lifestyle and made for implantation in older patients with limited activity in daily lifestyle. As healthcare around the world improves people are living longer and different ethnicities have different lifestyles and different knee anatomies. As a result, orthopedic surgeons are implanting younger patients for painful and arthritic degenerative changes of the knee and these patients are not willing to sacrifice their lifestyle with the limited range of motion given by the older implants. Limited range of motion is a current issue in knee replacement prosthesis technology.

When an orthopedic surgeon contemplates performing total knee arthroplasty for a painful arthritic knee, the main objective is to allow for painless walking. However, the remaining functions of a normal life, such as climbing stairs, getting up from a chair, ability to squat, and kneeling are not easily accomplished because of limitations on postoperative range of motion.

Currently, knee replacement designs used to enhance postoperative flexion of the knee use cam-post or posterior stabilization style bearing inserts. These types of designs have produced undesirable consequences, for example excessive wear or loosening of the femoral component. These undesirable consequences are due to the interaction of a post in the mid to posterior region of the knee. In addition, the cam-post implants do not accomplish roll back and thus will not allow natural movement of the knee. Many of the commercially available femoral implants have longer, thinner posterior condyles. The longer, thinner posterior condyles create lifting of the anterior compartment of the knee joint on flexion of greater than 90° because of impingement between the most posterior condyle tip and the tibial bearing component. In addition, the currently available implant designs do not allow for the patella to sit in its most anatomical position and direction, which also inhibits flexion.

The present invention for a new and improved femoral knee implant addresses the ongoing need to improve postoperative functionality through increase range of motion.

### SUMMARY OF THE INVENTION

In one aspect, provided herein is a femoral knee replacement prosthesis including a femoral component, a tibial bearing component, and a tibial platform component. The femoral component includes an anterior condyle with a proximal lateral aspect adjacent a proximal medial aspect separated by a patella groove. The femoral component also includes a distal condyle lateral aspect inferior the proximal lateral aspect of the anterior condyle and a distal condyle medial aspect inferior the proximal medial aspect of the anterior condyle, wherein the distal condyle medial aspect is adjacent the distal condyle lateral aspect. The femoral condyle further includes a lateral posterior condyle extending posteriorly from the distal condyle lateral aspect and a medial posterior condyle extending posteriorly from the distal condyle medial aspect, wherein the lateral posterior condyle is parallel to the medial posterior condyle. The tibial bearing component includes a proximal side for mating with the femoral component and a distal side with a stem. The tibial platform component includes a proximal side with an opening for receiving the tibial bearing component and a distal side with a post adapted to be fixed in a tibia.

In another aspect, provided herein is a femoral implant including an anterior planar surface, a posterior planar surface, a distal planar surface, an anterior-distal planar surface, a posterior-distal planar surface, and at least one post secured to the distal planar surface. The anterior planar surface opposes an anterior condyle with a proximal lateral aspect and a proximal medial aspect. The posterior planar surface is parallel to the anterior planar surface and opposes the posterior condyles. The distal planar surface opposes the distal condyles and is angled distally to form a perpendicular line connecting the anterior planar surface and the posterior planar surface at an angle of approximately 15°. The anterior-distal planar surface opposes the anterior condyle and the distal condyles and connects a distal end of the anterior planar surface and an anterior end of the distal planar surface. The posterior-distal planar surface opposes the distal condyles and posterior condyles and connects a posterior end of the distal planar surface and a distal end of the posterior planar surface.

The various embodiments of the invention replace the painful and deformed knee joint with the artificial knee implant prosthesis of the present invention. The invention will restore normal function, full flexion of the knee up to about 160 degrees, eliminate pain, and last one's lifetime. Further, the present invention is designed to achieve near normal function enabling the patient to return to their everyday activities.

These, and other objects, features and advantages of this invention will become apparent from the following detailed description of the various aspects of the invention taken in conjunction with the accompanying drawings.

### BRIEF DESCRIPTION OF DRAWINGS

The accompanying drawings, which are incorporated in and constitute a part of the specification, illustrate embodiments of the invention and together with the detailed description herein, serve to explain the principles of the invention. The drawings are only for purposes of illustrating preferred embodiments and are not to be construed as limiting the invention.

FIG. 1 shows an anterior oblique view of a femoral knee prosthesis minus the patella from the medial side, in accordance with an aspect of the present invention;

FIG. 2 shows exploded view of the femoral knee prosthesis of FIG. 1 from posterior lateral side, in accordance with an aspect of the present invention;

FIG. 3 shows the inferior view of the femoral component of the femoral knee prosthesis of FIG. 1, in accordance with an aspect of the present invention;

FIG. 4 shows a front view of the femoral component of the femoral knee prosthesis of FIG. 1, in accordance with an aspect of the present invention;

FIG. 5 shows a superior view of the tibial bearing component of the tibial component of the femoral knee prosthesis of FIG. 1, in accordance with an aspect of the present invention;

FIG. 6 shows a front view of the tibial bearing component of FIG. 5, in accordance with an aspect of the present invention;

FIG. 7 shows a side view of the tibial bearing component of FIG. 5, in accordance with an aspect of the present invention;

FIG. 8 shows an inferior view of the tibial bearing component of FIG. 5, in accordance with an aspect of the present invention;

FIG. 9 shows an isometric view of the tibial bearing component of FIG. 5, in accordance with an aspect of the present invention;

FIG. 10 shows a superior view of the tibial tray component of the femoral knee prosthesis of FIG. 1, in accordance with an aspect of the present invention;

FIG. 11 shows a side view of the tibial tray component of FIG. 10, in accordance with an aspect of the present invention;

FIG. 12 shows a front view of the tibial tray component of FIG. 10, in accordance with an aspect of the present invention;

FIG. 13 shows a lateral side view of the femoral component of the femoral knee prosthesis of FIG. 1, in accordance with an aspect of the present invention;

FIG. 14 shows the lateral side view of the femoral component of FIG. 13 depicting the angles of three circular zones, in accordance with an aspect of the present invention;

FIG. 15 shows the lateral side view of the femoral component of FIG. 13 depicting a midpoint of the inner posterior condyle surface, in accordance with an aspect of the present invention;

FIG. 16 shows a lateral view of the posterior condyle of the femoral component of FIGS. 13 and 14 depicting the radius of the posterior condyle of the present invention, in accordance with an aspect of the present invention;

FIG. 17 shows a lateral view of the assembled femoral knee prosthesis of FIG. 1, in accordance with an aspect of the present invention;

FIG. 18 shows a lateral view of the assembled femoral knee prosthesis of FIG. 1 in 60° of flexion, in accordance with an aspect of the present invention;

FIG. 19 shows a lateral view of the assembled femoral knee prosthesis of FIG. 1 in 90° of flexion, in accordance with an aspect of the present invention;

FIG. 20 shows a lateral view of the assembled femoral knee prosthesis of FIG. 1 in 130° of flexion, in accordance with an aspect of the present invention; and

FIG. 21 shows a lateral view of the assembled femoral knee prosthesis of FIG. 1 in 120°, 130°, and 140° of flexion, in accordance with an aspect of the present invention; and

FIG. 22 shows a lateral view of the assembled femoral knee prosthesis of FIG. 1 in 160° of flexion, in accordance with an aspect of the present invention.

### DETAILED DESCRIPTION FOR CARRYING OUT THE INVENTION

In this application, the words proximal, distal, anterior, posterior, medial, lateral, superior and inferior are defined by their standard usage for indicating a particular part or portion of a bone or prosthesis coupled thereto, or directional terms of reference, according to the relative disposition of the natural bone. For example, “proximal” means the portion of a bone or prosthesis nearest the torso, while “distal” indicates the portion of the bone or prosthesis farthest from the torso. As an example of directional usage of the terms, “anterior” refers to a direction towards the front side of the body, “posterior” refers to a direction towards the back side of the body, “medial” refers to a direction towards the midline of the body and “lateral” refers to a direction towards the sides or away from the midline of the body. In addition, as an example of directional usage of the terms, “superior” refers to a direction towards the top of the body or head and “inferior” refers to a direction towards to bottom of the body or feet. Such terms are well understood in describing the orientation of the implant and normal anatomy of the knee.

Further, the devices, methods, and the aspects, components, features and the like thereof, disclosed herein are described with respect to one side of the body for brevity purposes. However, as the human body is relatively symmetrical or mirrored about a line of symmetry (midline), it is hereby expressly contemplated that the devices, methods, and the aspects, components, features and the like thereof, described and/or illustrated herein may be changed, varied, modified, reconfigured or otherwise altered for use or association with another side of the body for a same or similar purpose without departing from the spirit and scope of the invention.

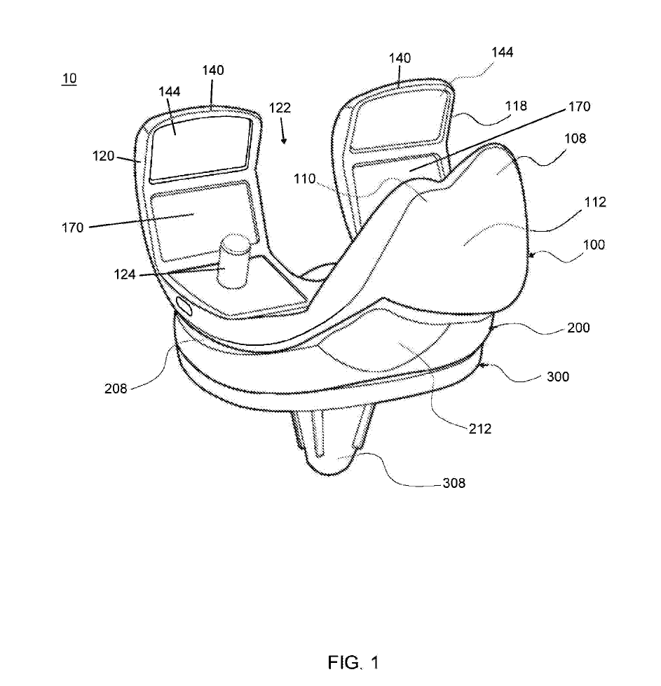

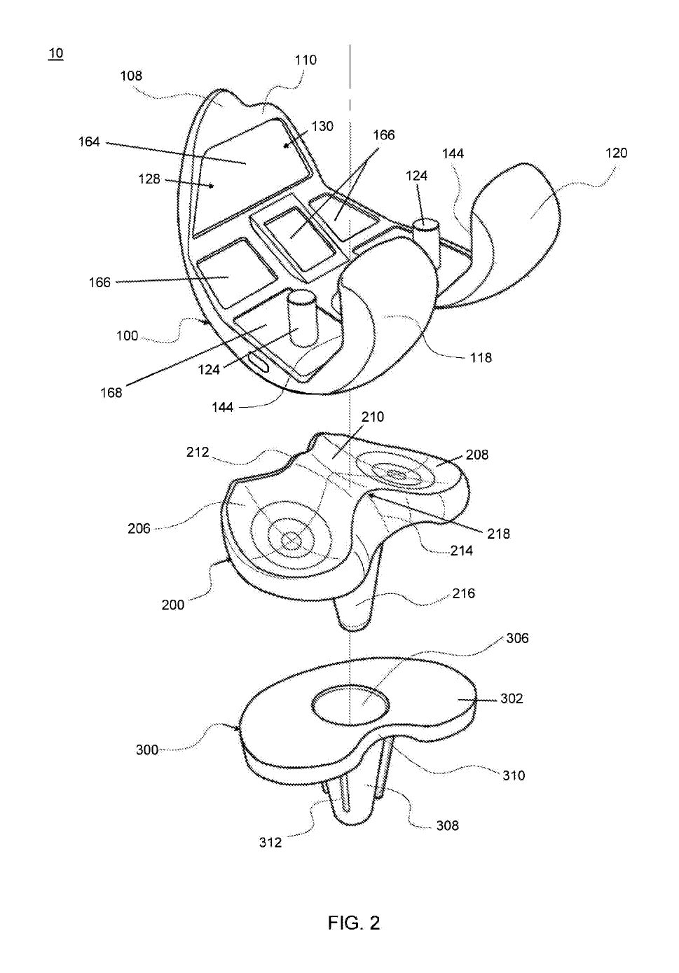

Referring to the drawings, wherein like reference numerals are used to indicate like or analogous components throughout the several views, and with particular reference to FIGS. 1 and 2, there is illustrated an exemplary embodiment femoral knee prosthesis 10. The terms “femoral knee prosthesis,” “implanted knee,” “implant,” “femur-tibial implant,” and “hyperflexion implant” are used interchangeably and refer to a device for replacement of a damaged knee joint. As best seen in FIG. 1, the femoral knee prosthesis 10 is assembled and shown from an anterior-medial view. The femoral knee prosthesis 10 includes a femoral component 100, a tibial bearing component 200, and a tibial platform 300. The femoral component 100 sits on the proximal side of the tibial bearing component 200 while the distal side of the tibial bearing component 200 sits on a proximal surface of a tibial platform 300. An exploded view of the femoral knee prosthesis 10 is illustrated in FIG. 2 and shows the femoral component 100, tibial bearing component 200, and tibial platform 300 of the present embodiment from a lateral superior view.

The femoral component 100 is adapted to be fixed to the distal end of a femur. The femoral component 100 includes an anterior condyle or phalange 102 connected to distal condyles 104, which are in turn connected to posterior condyles 106. The anterior condyle 102 includes a proximal lateral aspect 108 adjacent a proximal medial aspect 110 and a patella groove 112 situated between the lateral aspect 108 and the medial aspect 110 to accommodate the patella. The patella groove 112 continues into the distal condyles 104 which include a distal lateral aspect 114 adjacent a distal medial aspect 116, wherein the patella groove 112 runs between the distal lateral aspect 114 and the distal medial aspect 116. The posterior condyles 106 include a lateral posterior condyle 118 parallel to a medial posterior condyle 120, and an intercondylar opening 122 between the lateral posterior condyle 118 and the medial posterior condyle 120. The interior surface of the distal lateral aspect 114 and the distal medial aspect 116 each include a stem or post 124 for attachment or securement of the femoral component 100 to a patient's femur.

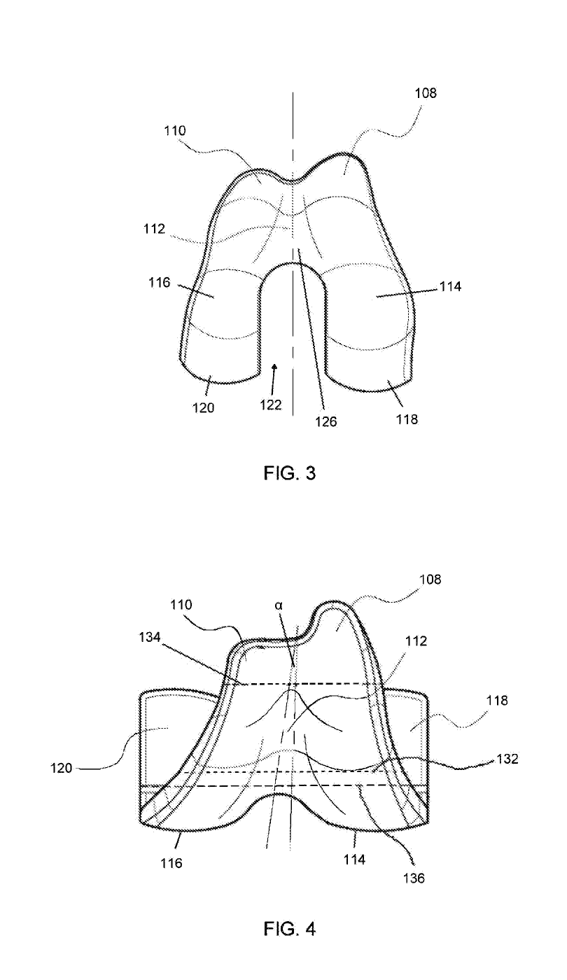

Referring now to FIGS. 3, 4, and 13, with continued reference to FIGS. 1 and 2, the proximal lateral aspect 108 extends further in a proximal direction than the proximal medial aspect 110 to cover the exposed osteotomy surface of the femur. The proximal medial aspect 110 is about thirty percent (30%) shorter than the proximal lateral aspect 108, wherein the proximal medial aspect 110 may have a proximal to distal dimension or height from the inferior side of the distal condyles 104 to the superior end of the proximal medial aspect 110 ranging from approximately 41 to 52 mm and the proximal lateral aspect 108 may have a proximal to distal dimension or height from the inferior side of the distal condyles 104 to the superior end of the proximal lateral aspect 108 ranging from approximately 44 to 62 mm. The interior surface 128 of the proximal lateral aspect 108 may also have an anterior planar surface 164 of the anterior condyle 102 with a height ranging from approximately 29 to 39 mm, while the anterior planar surface 164 of the proximal medial aspect 110 of the anterior condyle 102 with a height ranging from approximately 20 to 29 mm. In the present invention, the proximal lateral aspect 108 may be thicker than the proximal medial aspect 110 to provide stability of the patella component as the patella travels during acute flexion of the knee. The proximal lateral aspect 108 may have an anterior-posterior dimension or thickness ranging from approximately 7 to 12 mm and the proximal medial aspect 110 may have an anterior-posterior dimension or thickness ranging from approximately 4 to 5 mm. By increasing the thickness of the proximal lateral aspect 108 relative to the proximal medial aspect 110 in an anterior-posterior direction by about 2 to 7 mm the patella groove 112 is deepened longitudinally to accommodate the sinking of the patella as the knee flexes. The patella groove 112 may enhance flexion of the knee.

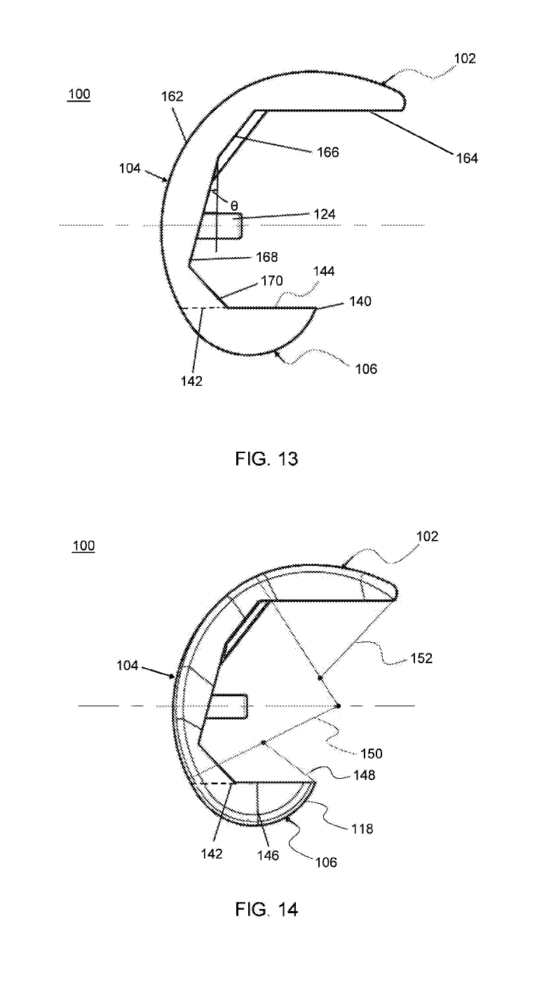

The patella groove 112 may have an angle α, which generally matches the patella implant and is approximately 6°. The angle α may also be directed 6° lateral to the central angle of the distal femur to mimic the anatomical patellar groove. The medial-lateral dimension or width of the anterior phalange 102 at the anterior intercondylar region 132 may range from approximately 38 to 54 mm. The width of the anterior intercondylar region 132 is measured on the exterior surface of the femoral component 100 at a position equivalent to where the anterior and posterior cruciate ligaments would be attached to the interior surface of the femur. Referring now to FIG. 13, the proximal lateral aspect 108 of the anterior phalange 102 may also include a proximal-distal dimension or height from the point the anterior intercondylar width is measured to the superior end of the proximal lateral aspect 108 of the anterior phalange 102 that may range from about 29 to 39 mm. In addition, the proximal medial aspect 110 of the anterior phalange 102 may include a proximal-distal dimension or height from the point the anterior intercondylar width is measured to the superior end of the proximal medial aspect 110 ranging from about 20 to 29 mm. As seen in FIG. 4, the anterior phalange 102 may also include a medial-lateral dimension or width taken at reference line 134 ranging from about 29 to 41 mm.

As seen in FIGS. 3 and 4, the distal lateral condyle 114 and the distal medial condyle 116 of the two distal condyles 104 are a first and second bearing surface, respectively, for the femoral component 100 against the tibial bearing component 200. The distal lateral condyle 114 and the distal medial condyle 116 are connected by the distal portion of the patella groove 112 and an intercondylar portion 126. As illustrated in FIG. 13, the distal condyles 104 may have an anterior-posterior exterior length from the exterior surface of the anterior phalange 102 to the exterior surface of the two posterior condyles 118, 120 ranging from about 52 to 84 mm. The distal condyles 104 may also have an anterior-posterior interior length measured from the interior surface of the anterior phalange 102 to the interior surface of the two posterior condyles 118, 120 that ranges generally from approximately 35 to 55 mm. The distal condyles 104 may have a proximal to distal thickness of approximately 9 mm.

As shown in FIGS. 3 and 4, the medial-lateral dimension or width of the two posterior condyles 106 along the transepicondylar axis 136 may range from approximately 58 to 76 mm. The lateral posterior condyle 118 may be spaced apart from the medial posterior condyle 120 forming an intercondylar opening 122, which may have a medial-lateral dimension or width ranging from about 16 to 24 mm. The lateral posterior condyle 118 and the medial posterior condyle 120 may have a proximal to distal dimension or height from the tips 140 of the posterior condyles 118, 120 to the exterior surface of the distal condyles 104 along line 142 ranging from approximately 36 to 42 mm, as shown in FIG. 13. The lateral and medial posterior condyles 118, 120 may have an anterior to posterior thickness measured at the midpoint 146 along line 142 and perpendicular to line 142, wherein the thickness may range from about 10 to 17 mm. As seen in FIGS. 13 and 14, the midpoint 146 is defined when bisecting the line drawn from the tip 140 of the lateral or medial posterior condyle 118, 120 to its linear extension on the curved external surface of the distal condyles 104. The femoral knee prosthesis 10 of the present invention has increased flexion as a result of increasing the thickness of the midpoint 146 of the posterior condyles 106.

As illustrated in FIGS. 1, 2, and 13, an anterior-posterior box is formed on the inner surface of the femoral component 100 opposing the anterior condyle, distal condyles, and posterior condyles. The anterior-posterior box includes five planar surfaces, an anterior surface 164, an anterior-distal surface 166, a distal surface 168, a posterior-distal surface 170, and a posterior surface 144. The anterior planar surface 164 opposes the anterior condyle 102 creating a thickness of the proximal lateral aspect 108 of approximately 7 mm to 12 mm and a thickness of the proximal medial aspect 110 of approximately 4 mm to 5 mm. The anterior planar surface 164 has a height on the proximal lateral aspect 108 ranging from about 29 mm to 39 mm and a height on the proximal medial aspect 110 ranging from about 20 mm to 29 mm. The distal planar surface 168 opposes the distal condyles 104 creating a thickness of the distal condyles of approximately 9 mm. The anterior-distal planar surface 166 opposes both the anterior condyle 102 and the distal condyle 104 and connects a distal end of the anterior planar surface 164 and an anterior end of the distal planar surface 168. The posterior planar surface 144 opposes the posterior condyles 106 creating a thickness of the lateral and medial posterior condyles 118, 120 ranging from approximately 10 mm to 17 mm. The posterior planar surface 144 has proximal-distal dimension or height of the lateral and medial posterior condyles 118, 120 ranging from approximately 14 to 20 mm. The posterior-distal planar surface 170 opposes both the distal condyles 104 and the posterior condyles 106 and connects a posterior end of the distal planar surface 168 and a distal end of the posterior planar surface 144. The length between the anterior planar surface 164 and the posterior planar surface 144 ranges from approximately 35 mm to 55 mm. The distal planar surface 168 may also be angled distally from a perpendicular line connecting the anterior planar surface 164 and posterior planar surface 144 at an angle θ of approximately 15°.

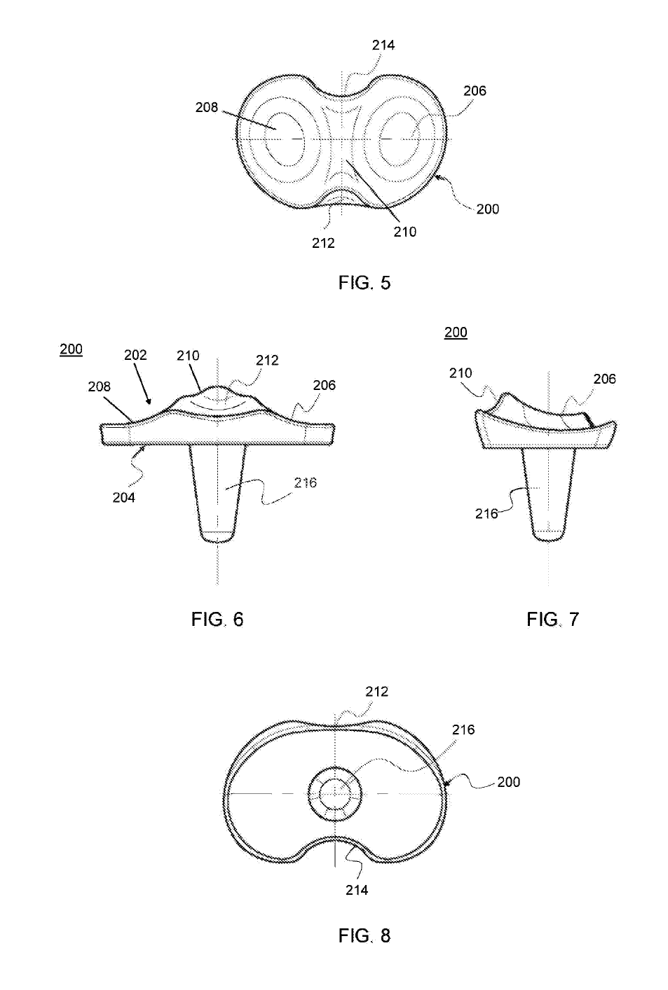

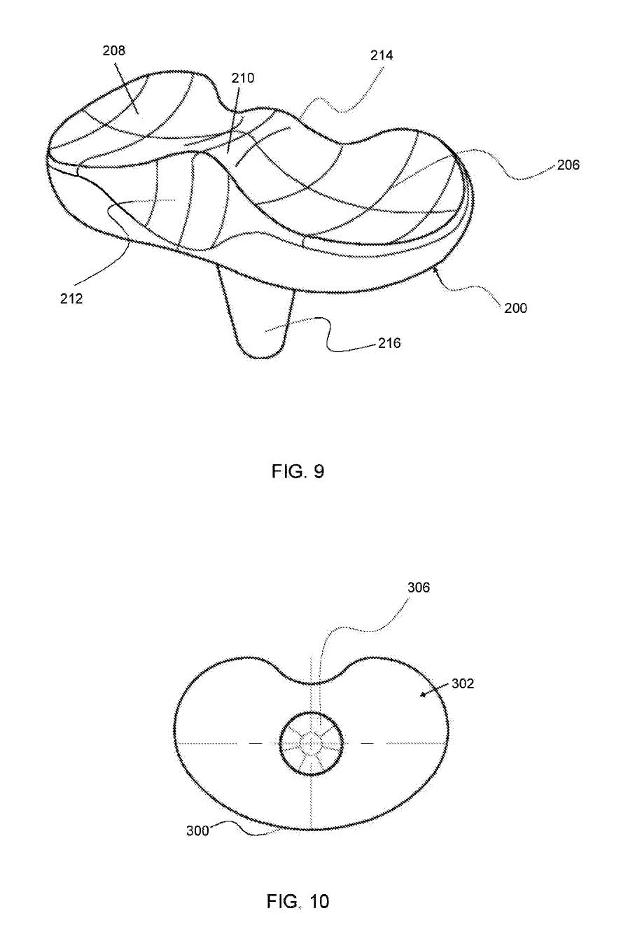

As illustrated in FIGS. 1-2 and 5-9, the tibial bearing component 200 includes a proximal side 202 and a distal side 204. The tibial bearing component 200 may be comprised of a bio-compatible bearing material, such as for example UHMWPE. The tibial bearing component 200 is shaped to enable the femoral component 100 to articulate with the tibial bearing component 200, which is mobile and rotating in nature. The proximal side 202 includes a lateral depression 206 parallel to a medial depression 208 and a central prominence 210 between the lateral and medial depressions 206, 208. The lateral and medial depressions 206, 208 are deepened concave surfaces shaped to accommodate the convex outer bearing surfaces of the distal condyles 104 and posterior condyles 106 of the femoral component 100. The corresponding concave shape of the lateral and medial depressions 206, 208 maximizes the area of contact between the femoral component 100 and the tibial bearing component 200. In addition, by maintaining the large area of contact between the distal and posterior condyles 104 and 106 and the tibial bearing component 200, the mobile bearing design of the implant 10 is able to provide a natural rotation of the tibial bearing component 200 in extension and flexion. The central prominence 210 provides additional stabilization of the tibial bearing component 200 to prevent medial and lateral rocking of the implanted prosthesis 10 and to provide increased contact area. The anterior side of the central prominence 210 includes an anterior groove 212 and the posterior side of the central prominence 210 includes a posterior groove 214. The anterior groove 212 is shaped to avoid any undue contact with a patella in deep flexion. The posterior groove 214 is shaped to accommodate a retained posterior cruciate ligament (“PCL”). The distal side 204 of the tibial bearing component 200 has a generally planar surface and includes a stem 216 extending from the generally planar surface in a distal direction.

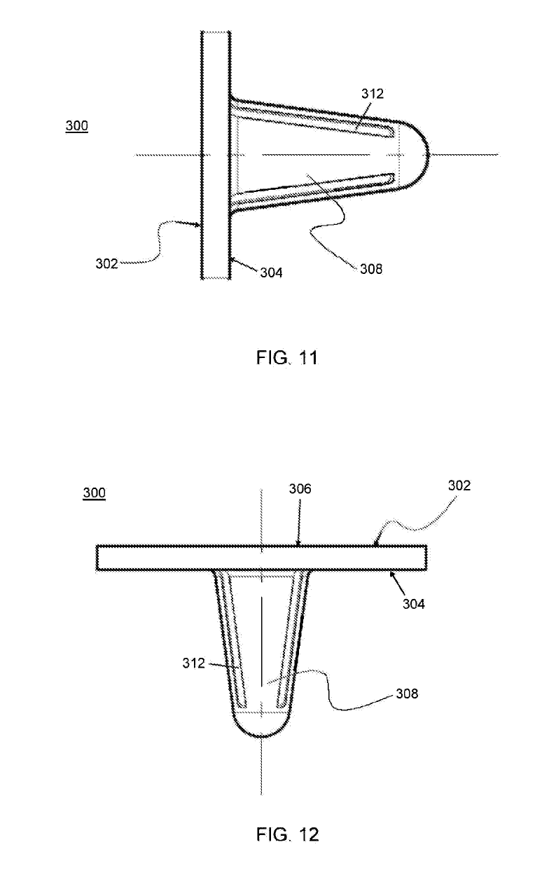

The tibial platform or tray 300, as shown in FIGS. 1-2 and 10-12, includes a proximal side 302 and a distal side 304. The proximal side 302 has a generally planar surface that allows for rotation of the tibial bearing component 200. The proximal side 302 also includes an opening 306, which is generally centered in the medial-lateral direction. The opening 306 enables the tibial platform 300 to articulate with the stem 216 of the tibial bearing component 200 when the stem 216 is inserted into the opening 306. The distal side 304 has a generally planar surface and includes a stem 308 for securing to the tibia. The stem 308 is located generally central of the tibial platform 300 and may include at least one fin or rib 312. In the illustrated embodiments, the at least one fin or rib 312 includes four fins. However the at least one fin 312 may preferably be between 2 and 6 fins and is more preferably four fins. The at least one fin 312 may prevent rotation of the tibial platform 300 after it is fixed in the tibial bone. In addition, the stem 308 may also prevent and accommodate any undue rocking at the tibia during weight bearing activities of the knee. The posterior side of the tibial platform 300 includes a posterior groove 310. The posterior groove 310 is nearly identical to the posterior groove 214 of the tibial bearing component 200 and is similarly shaped to accommodate a retained PCL.

The femoral component 100, specifically the posterior condyles 106, are configured to inhibit spin-out and enhance full flexion of the implanted knee, to, for example approximately 160 degrees. Spin-out of the tibial component 200 is prevented by tightening the collateral ligaments in flexion by the lifting of the femur with the increased thickness of the posterior condyles 106. The posterior condyle 106 will also compress the femoral component 100 onto the tibial bearing component 200, thereby, stabilizing the femoral component 100 relative to the tibial component 200. The femoral knee prosthesis 10 of the present invention also enhances full flexion by creating a wider posterior gap, which enables the femoral component 100 to roll back freely and create the optimal patella tension and allowing the implanted knee prosthesis 10 to flex fully.

The femoral knee prosthesis 10 of the present invention will ensure the ability for hyperflexion by enhancing and stabilizing proper roll back, creating a wider bearing contact area in deep flexion, which reduces excessive wear and posterior stability, allowing for a more deeply seated and naturally oriented patellar tendon, and creating the proper width between the trans-epicondylar 136 and anterior intercondylar 132. All these factors contribute to the implanted knee prosthesis' ability to achieve hyperflexion (i.e. 160 degrees). The femoral knee prosthesis 10 lifts the posterior condyles 106 in acute flexion (i.e. 160 degrees). As shown in FIG. 16, lifting the posterior compartment of the knee is accomplished with a femoral component 100 which includes a shortened proximal-distal height 142 with a rounded off the end 140, as well as an increased thickness of the anterior-posterior diameter of the circle 160. The additional thickness of the lateral and medial posterior condyles 118, 120 is maximized in the midpoint 146 of the lateral and medial posterior condyles 118, 120. The additional thickness at the midpoint 146 is not intended to accommodate any instability in posterior flexion gap. By increasing the thickness of the lateral and medial posterior condyles 118, 120 and proximal lateral aspect 108 of the anterior condyle 102, the over-all anterior-posterior length of the femoral component 100 will increase and allow for hyperflexion of the knee (i.e. 160 degrees).

The increased anterior-posterior diameter of circle 160 of the posterior condyles 106 also allows for the posterior space of the implanted knee 10 to greatly increase during flexion allowing the tibial bearing component 200 of the implanted knee 10 to flex to full or acute flexion, approximately 160 degrees, without impingement at the posterior of the knee between the posterior rim 218 of the tibial bearing component 200 and the lateral and medial posterior condyles 118, 120. In normal knees, as one flexes the knee fully, for example, to squat or kneel, the posterior portion of the normal tibia slides under the posterior condyle of the femur as it rolls back and allows the knee to fully flex. This embodiment of the design of the femoral component 100 precisely mimics the biomechanics of a normal knee flexion up to 160 degrees. The increased anterior-posterior diameter 160 of the posterior condyles 106 also allows for proper alignment and tension of the patella.

Referring now to FIGS. 13-15, side views of the femoral component 100 are shown. In vivo, Asian posterior condyles are typically 14 to 20 mm in length and Caucasian are greater than 20 mm in length. The posterior planar surface 144 of the posterior condyles 106 has a height, ranging from approximately 14 to 20 mm, to accommodate extension of the lateral posterior condyle 118 and medial posterior condyle 120 of the femoral implant 100. The anterior posterior curvature of the lateral and medial posterior condyles 118, 120 is configured to rotate around the posterior radius of curvature 148 which has a smaller single axis of rotation when the knee flexes. The posterior radius 148 of the bearing surface of the posterior condyles 106 ranges from approximately 14 to 20 mm.

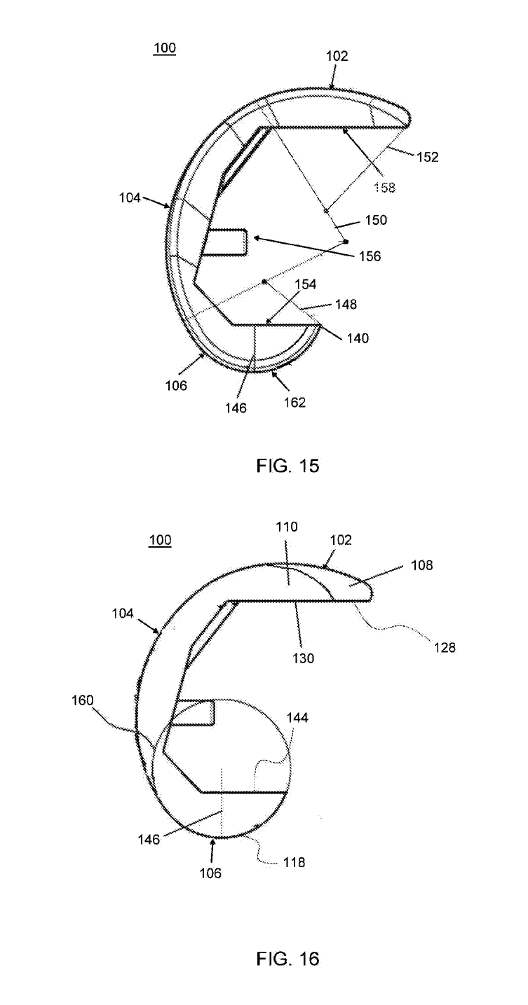

A lateral view of the femoral component 100 showing the separation of the zones in different angles is depicted in FIGS. 14 and 15. The femoral component 100 is designed with three main articulating zones. The posterior articulating zone 154 will create the smallest arc of curvature for the bearing surface of the posterior condyles 106. The arc of curvature for the shortened and thickened condyles enables the femoral component 100 to flex up to 160 degrees. The posterior zone 154 will have a posterior radius 148 of about 14 to 20 mm. The distal articulating zone 156 has the largest radius of curvature and is shown as the bearing surface of the distal condyles 104. The posterior zone 154 is configured as a convex shaped large contact area to decrease contact stress and increase stability. The distal zone 156 will have a distal radius 150 of about 32 to 42 mm. The most anterior articulating zone 158 will be a gentle curve creating a medium sized circle using the exterior of the anterior condyle 102 and having an anterior radius 152 of about 23 to 29 mm. The anterior zone 158 created by the anterior radius 152 will have a gentle curve with the center of motion in the anterior portion of the knee. The distal zone 156 created by the distal radius 150 will have the center (so-called instant center) slightly posterior, maintaining the posterior momentum at the initiation of flexion of the knee. The posterior zone 154 created by the posterior radius 148 enables the implanted knee to flex fully close to approximately 160 degrees. Several advantages of the present invention include: (1) eliminating the possibility of impingement of the posterior part of the distal femur in acute flexion (160 degrees) by the posterior end of tibial bearing component 200, (2) allowing the tibial bearing component 200 to roll-back smoothly like a normal knee in flexion, (3) allowing the tibial bearing component 200 to rotate normally to let the soft tissues surrounding the knee accommodate full flexion without undue stress for natural and smooth maximum flexion, and (4) allowing the patellar tendon to deeply seat in the implant 10 and thus allow more flexion.

FIG. 16 shows a circle 160, which outlines the profile of the posterior condyles 106 of the knee. The circle 160, that forms the exterior surface 162 of the lateral posterior condyle 118 of the present invention, has a radius of approximately 14 to 20 mm. The circle 160 represents the range of motion for the femoral component 100. Again, the thickness of the posterior condyles 106 is thicker at the midpoint 146 which ranges from approximately 10 to 17 mm. The circle 160 exhibits; (1) a smaller single axis to enhance flexion of the knee, (2) a smaller single axis to accelerate the flexion, readily making the knee easy to flex naturally and normally, (3) by increasing the height of the femoral component (anterior posterior “AP” alignment), increases the sum of the vector of the anterior extensor mechanism and thus increases the strength of the extension power of the knee. In a healthy knee, the vector is formed by the patellar ligament, patella and quadriceps structure. The sum of the vector is the actual strength of the extension power of the knee. Since the portion of vector of the patellar ligament and patella is fixed, the variance of the power of the extension of the knee, for activities such as, chair rise, stair climbing, etc., depends on the strength of the quadriceps structure. Typically the quadriceps structure is weakened by long standing pain and disuse. By lengthening the vector and increasing the overall anterior posterior lengths of the femoral component 100 the muscle function will be enhanced post-operatively. Further, in the present invention in acute flexion, the posterior condyles 106 will clear the tibial bearing component 200 and allow the tibial bearing component 200 to roll back.

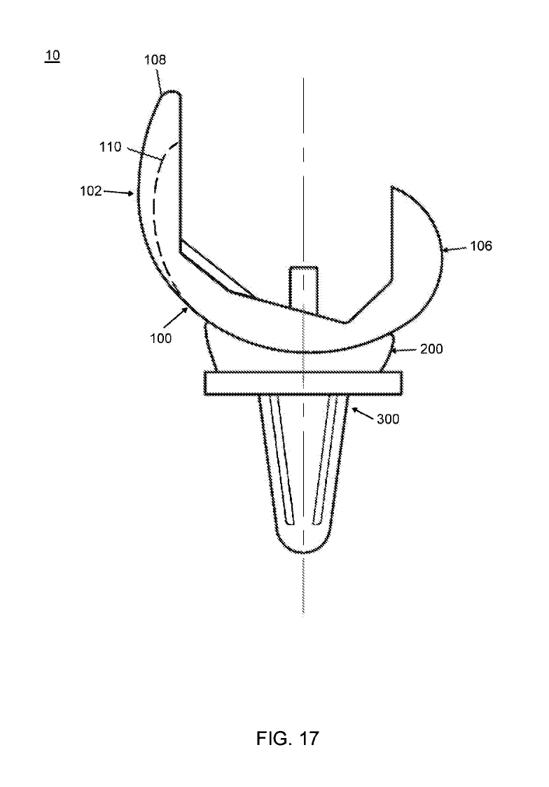

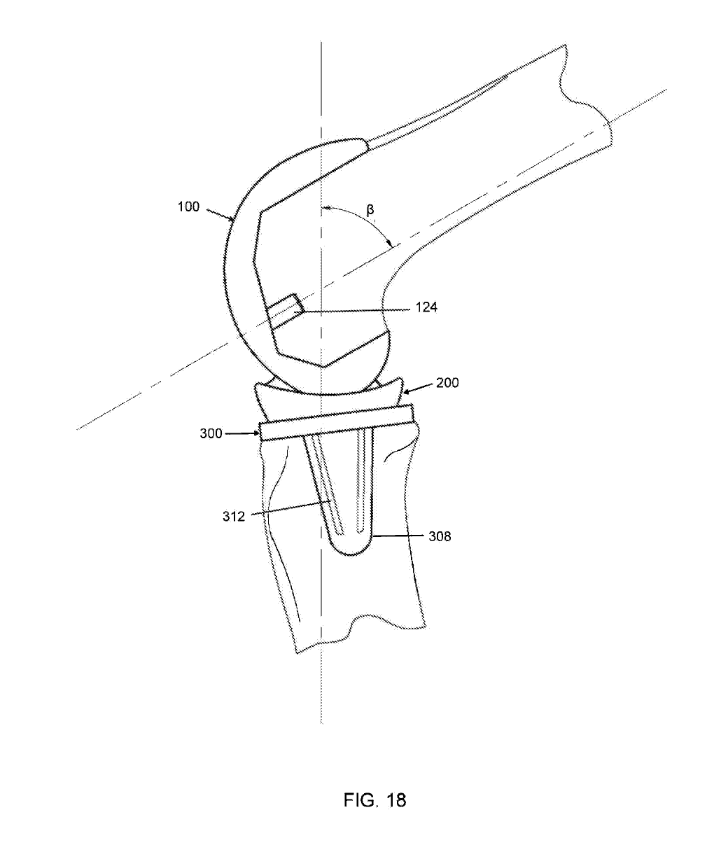

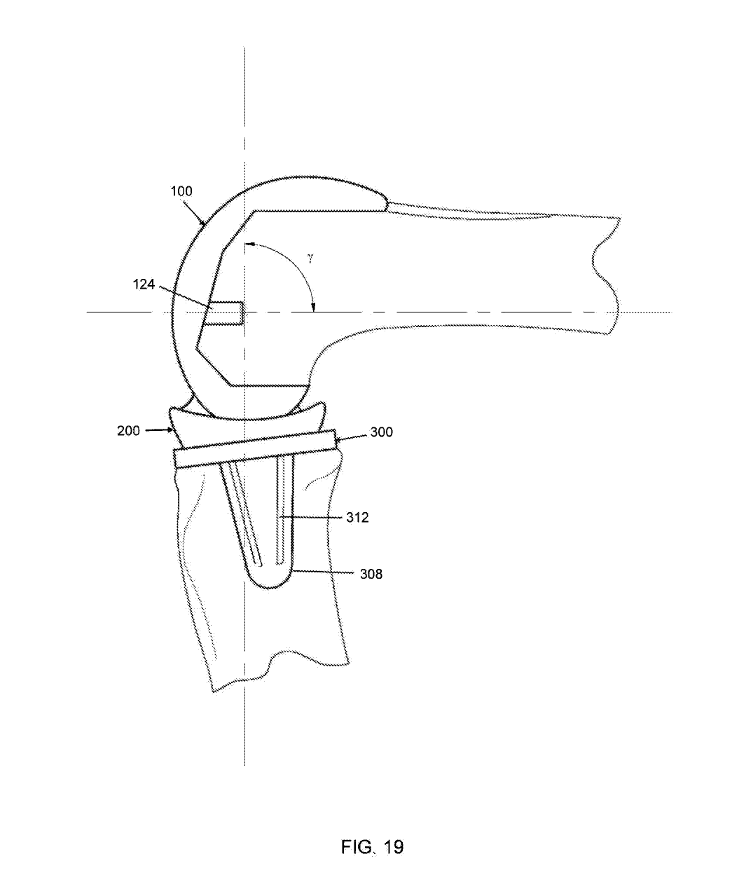

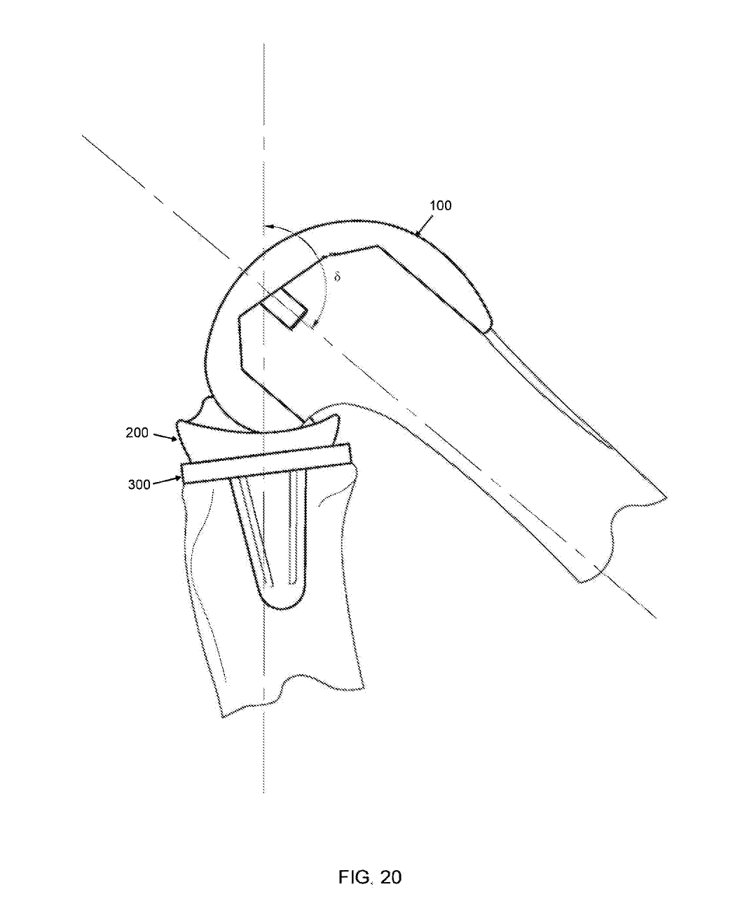

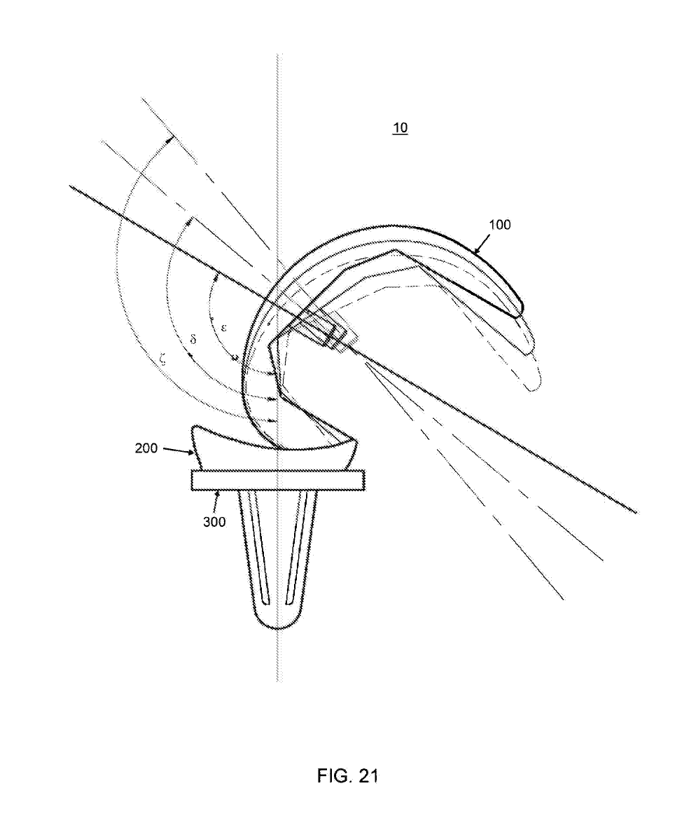

Referring now to FIGS. 17-22 which are lateral views of the femoral knee prosthesis 10 in various degrees of flexion, the femoral component 100 is shown in relation to the tibial bearing component 200 and the tibial platform 300 in angles ranging from approximately 0 to 160 degrees. The FIGS. 17-22 demonstrate how the implanted knee 10 will behave in flexion. Referring to FIG. 17, the femoral knee prosthesis 10 is illustrated at about 0 degrees of flexion. At an angle β of about 60 degrees, the tibial bearing component 200 will rollback, maintaining full contact in extension and a wide area of contact as shown in FIG. 18. This configuration of the wide area contact will diminish the contact stress between the femoral component 100 and tibial bearing component 200. The longevity of the wearable bearing material is increased because of the diminished contact stress. The concept of increased contact area with decrease contact stress has proven its longevity in vivo and in vitro tests. FIG. 19 depicts the implanted knee 10 in flexion at an angle γ of approximately 90 degrees, while FIG. 20 shows the implanted knee 10 in flexion at an angle δ of about 130 degrees of flexion. Referring now to FIG. 21, the femoral knee prosthesis 10 is illustrated in a range of degrees of flexion including an angle ε of about 120 degrees, the angle 6 of about 130 degrees, and an angle ζ of about 140 degrees.

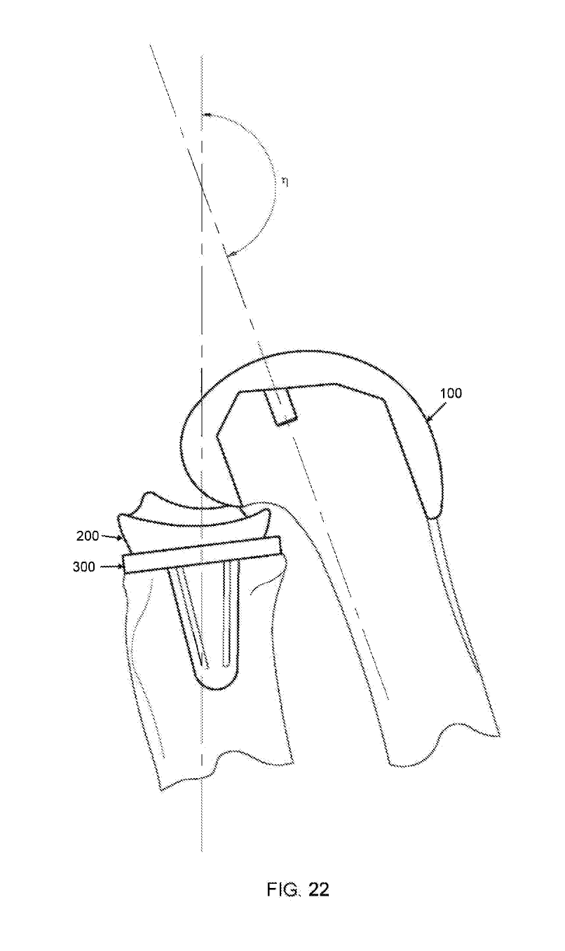

Finally, the implanted knee 10 is depicted in an angle η of approximately 160 degrees of flexion in FIG. 22. Rollback is important for enhanced maximal flexion of the implanted knee 10 to nearly 160 degrees and reduction of bearing material wear of the tibial bearing component 200. The implant 10 will rotate as the implanted knee flexes. The medial portion of the tibial bearing component 200 is the axis of rotation as the lateral portion moves back in a posterior direction. The sum of this rotation may occur at the femoral-tibial contact area as well as the flat portion of the tibial tray 300. The combined rotation occurs due to the design of the mobile bearing enabling movement of the tibial bearing component 200 in the tibial platform 300 and the action of the soft tissue structure of the knee. Of course, the soft tissue structures (ligaments) need to be balanced at the time of the surgery.

The ratio between trans-epicondylar width 136 and anterior intercondylar width 132, as shown in FIG. 4, is also important. The true anterior intercondylar width 132 is formed after bone cuts of the femur have been made. The ratio of the trans-epicondylar width 136 and the anterior intercondylar width 132 of the femoral component 100 is a range of 100/70 to 100/72. The surrounding soft tissue will permit maximal and acute flexion, up to 160 degrees by having a ratio of 100/70-72.

The invention has been described with reference to the preferred embodiments. It will be understood that the architectural and operational embodiments described herein are exemplary of a plurality of possible arrangements to provide the same general features, characteristics, and general system operation. Modifications and alterations will occur to others upon a reading and understanding of the preceding detailed description. It is intended that the invention be construed as including all such modifications and alterations.

Having thus described the preferred embodiments, the invention is now claimed to be:

1. A femoral knee replacement prosthesis, comprising:
a femoral component comprising:
an anterior condyle having a proximal lateral aspect adjacent a proximal medial aspect separated by a patella groove;
a distal condyle lateral aspect inferior the proximal lateral aspect of the anterior condyle;
a distal condyle medial aspect inferior the proximal lateral aspect of the anterior condyle, wherein the distal condyle medial aspect is adjacent the distal condyle lateral aspect;
a lateral posterior condyle extending posteriorly from the distal condyle lateral aspect; and
a medial posterior condyle extending posteriorly from the distal condyle medial aspect, wherein the lateral posterior condyle is parallel the medial posterior condyle;
a tibial bearing component with a proximal side and distal side, wherein the proximal side mates with the femoral component and the distal side includes a stem; and
a tibial platform component with a proximal surface and a distal surface, the proximal surface mates with the distal side of the tibial bearing component and includes an opening for receiving the post of the tibial bearing component and the distal surface includes a post adapted to be fixed in a tibia.

2. The femoral knee replacement prosthesis of claim 1, wherein the distal condyles further comprises:
a first post on an interior surface of the distal condyle lateral aspect; and
a second post on an interior surface of the distal condyle medial aspect.

3. The femoral knee replacement prosthesis of claim 2, wherein the first post and the second post are adapted to be fixed into a femur.

4. The femoral knee replacement prosthesis of claim 1, wherein the lateral posterior condyle and the medial posterior condyle have an anterior-posterior height ranging from about 36 to 42 millimeters.

5. The femoral knee replacement prosthesis of claim 1, further comprising:
a midpoint in each of the lateral posterior condyle and the medial posterior condyle having a thickness ranging from about 10 to 17 millimeters.

6. The femoral knee replacement prosthesis of claim 1, wherein a posterior surface of an interior surface of the lateral and medial posterior condyles ranges from about 14 to 20 millimeters.

7. The femoral knee replacement prosthesis of claim 1, wherein the proximal lateral aspect of the anterior condyle has a proximal-distal height ranging from about 44 to 62 millimeters and an interior anterior surface height ranging from about 29 to 39 millimeters.

8. The femoral knee replacement prosthesis of claim 7, wherein the proximal medial aspect of the anterior condyle has a proximal-distal height ranging from about 41 to 52 millimeters and an interior anterior surface height ranging from about 20 to 29 millimeters.

9. The femoral knee replacement prosthesis of claim 1, wherein the proximal lateral aspect includes a first anterior-posterior thickness and the proximal medial aspect includes a second anterior-posterior thickness.

10. The femoral knee replacement prosthesis of claim 9, wherein the first anterior-posterior thickness is larger than the second anterior-posterior thickness.

11. The femoral knee replacement prosthesis of claim 10, wherein the first anterior-posterior thickness ranges from approximately 7 to 12 millimeters and the second anterior-posterior thickness ranges from approximately 4 to 5 millimeters.

12. The femoral knee replacement prosthesis of claim 1, wherein the distal condyles lateral aspect and medial aspect each have an anterior-posterior exterior length ranging from about 52 to 84 millimeters.

13. The femoral knee replacement prosthesis of claim 1, wherein the distal condyles lateral aspect and medial aspect each have an anterior-posterior interior length ranging from about 35 to 55 millimeters and a proximal-distal thickness of about 9 millimeters.

14. The femoral knee replacement prosthesis of claim 1, wherein the femoral component includes a trans-epicondylar width and an anterior inter-condylar width.

15. The femoral knee replacement prosthesis of claim 13, wherein the ratio of the trans-epicondylar width to the anterior inter-condylar width ranges from approximately 100/70 to approximately 100/72.

16. The femoral knee replacement prosthesis of claim 13, wherein the trans-epicondylar width ranges from approximately 58 to 76 millimeters and the anterior inter-condylar width ranges from about 38 to 54 millimeters.

17. The femoral knee replacement prosthesis of claim 1, wherein the femoral component further comprises:
an anterior articulating zone having an anterior radius;
a distal articulating zone having a distal radius; and
a posterior articulating zone having a posterior radius, wherein the distal radius is larger than the anterior radius and the proximal radius is the smallest.

18. The femoral knee replacement prosthesis of claim 17, wherein the anterior radius ranges from about 23 to 29 millimeters, the distal radius ranges from about 32 to 42 millimeters, and the posterior radius ranges from about 14 to 20 millimeters to facilitate hyperflexion.

19. The femoral knee replacement prosthesis of claim 18, wherein hyperflexion includes a range of motion from approximately 0° to 160°.

20. A femoral implant, comprising:
an anterior planar surface opposing an anterior condyle with a proximal lateral aspect and a proximal medial aspect;
a posterior planar surface parallel to the anterior planar surface and opposing posterior condyles;
a distal planar surface opposing distal condyles and angled distally from a perpendicular line connecting the anterior planar surface and the posterior planar surface at an angle of approximately 15°;
an anterior-distal planar surface opposing the anterior condyle and the distal condyles, wherein the anterior-distal planar surface connects a distal end of the anterior planar surface and an anterior end of the distal planar surface;
a posterior-distal planar surface opposing the distal condyles and posterior condyles, wherein the posterior-distal planar surface connects a posterior end of the distal planar surface and a distal end of the posterior planar surface; and
at least one post secured to the distal planar surface.

21. The femoral implant of claim 20, wherein an anterior-posterior thickness between the anterior planar surface and the proximal lateral aspect of the anterior condyle ranges from about 7 to 12 millimeters, an anterior-posterior thickness between the anterior planar surface and the proximal medial aspect of the anterior condyle ranges from about 4 to 5 millimeters, an anterior-posterior thickness between the posterior planar surface and the posterior condyles ranges from about 10 to 17 millimeters, the proximal-distal thickness between the distal planar surface and the distal condyles is about 9 millimeters, and an interior length ranging from about 35 to 55 millimeters.
